(19) United States
(12) Patent Application Publication
(10) Pub. No.: US 2010/0311740 A1
(43) Pub. Date: Dec. 9, 2010

(54) IMIDAZOPYRIDINE DERIVATIVES AS ACID PUMP ANTAGONISTS

(76) Inventors: Mark James Bamford, Harlow, Essex (GB); Richard Leonard Elliott, Essex (GB); Gerard Martin Paul Giblin, Essex (GB); Antoinette Naylor, Essex (GB); Terence Aaron Chal, Essex (GB); Andrew Kenneth Takle, Essex (GB); Jason Witherington, Essex (GB)

Correspondence Address: GlaxoSmithKline;GLOBAL PATENTS -US, UW2220, P. O. BOX 1539, KING OF PRUSSIA, PA, 19406-0939, US

(21) Appl. No.: 11/993,084

(22) PCT Filed: Jun. 28, 2006
(86) PCT No.: PCT/EP2006/006410

§ 371 Date: Dec. 19, 2007

(30) Foreign Application Priority Data
Jun. 30, 2005 (GB) 0513423.4

(51) Int. Cl. A61K 31/5377; C07D 471/04; A61K 31/437; A61P 1/04; A61K 31/496

(52) U.S. Cl. 514/233.2; 546/121; 514/300; 544/362; 514/253.04; 544/127

(57) ABSTRACT

This invention relates to newly identified imidazopyridine compounds of formula (I), to the use of such compounds in treating GERD and the like.

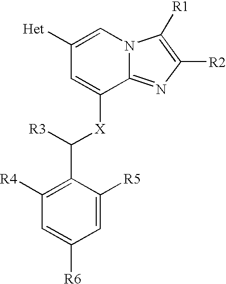

This invention relates to newly identified imidazopyridine compounds, to the use of such compounds in therapy and to their production.

The gastric H⁺/K⁺ ATPase, or proton pump, is responsible for gastric acid secretion from the acid secreting parietal cells of the stomach. The H⁺/K⁺ ATPase actively transports protons and K⁺ ions in opposite directions in a non-electrogenic manner, coupled to the hydrolysis of ATP. Under physiological conditions, this generates and maintains a proton gradient in excess of a million-fold across the luminal membrane of the gastric parietal cell.

Gastric acid is one of the major risk factors for gastrointestinal disease and specific inhibitors of the gastric H⁺/K⁺ ATPase are currently used for clinical treatments and control of hyperacidity. Such inhibitors can be classified into two groups, the first of which are the irreversible inhibitors such as omeprazole, which are termed proton pump inhibitors or PPIs. This class of compounds are weak bases which accumulate in the acidic canaliculi of active parietal cells where they rapidly form cationic tetracyclic sulphenamides. The sulphenamide then binds irreversibly to the lumenal surface of the H⁺/K⁺ ATPase and inhibits its activity.

In patients with gastro-oesophageal reflux disease (GERD), PPIs are currently the treatment of choice. However, the majority of patients treated with PPIs, histamine H2 receptor antagonists or prokinetic agents continue to experience frequent heartburn and nocturnal acid breakthough, suggesting that current therapies may not always achieve sufficient control of acid production.

Currently available PPIs may take 3-5 days to achieve maximal acid inhibition due to the fact that they require activation within the acidic canaliculus and thus target only actively secreting parietal cells. A proportion of the pumps therefore remains un-inhibited after each dose, and repeated daily dosing is required to reach a steady-state of inhibition.

The second group of H⁺/K⁺ ATPase inhibitors are the reversible inhibitors, which are described as acid pump antagonists (APAs) or potassium-competitive acid blockers (p-CABs). In contrast to the currently available PPIs, the reversible, K⁺ competitive APAs do not require activation in an acidic environment and block acid secretion in a direct manner by binding at or near the potassium binding site, resulting in a very rapid onset of action compared to PPIs. It is also expected that APAs will afford improvements in control of acid secretion over an extended period.

Accordingly the present invention provides a compound of formula (I)

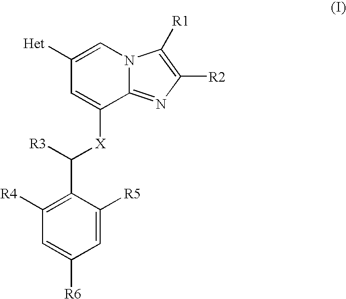

wherein X is NH, NR7 or O;
R1 is H, C₁₋₄ alkyl, CH₂CN, CH₂NH₂, C₃₋₆cycloalkyl, C₃₋₆cycloalkylC₁₋₄alkyl, C₂₋₄alkoxy, C₂₋₆alkenyl, C₂₋₆alkenyloxyC₁₋₄alkyl, C₂₋₆alkynyl, hydroxyC₁₋₄alkyl, C₁₋₄alkoxyC₁₋₄alkyl, hydroxyC₁₋₄alkoxyC₁₋₄alkyl, fluoroC₁₋₄alkyl, C₂₋₆alkynyloxyC₁₋₄alkyl, C₁₋₄alkylsulphonylC₁₋₄alkyl or NR8R9, where each of R8 and R9, which may be the same or different, are H or C₁₋₄alkyl or, together with the nitrogen to which they are attached, form a 5- or 6-membered heterocyclic group containing 0 to 3 further heteroatoms selected from N, O and S;
R2 is C₁₋₄alkyl, NH₂, C₃₋₆cycloalkyl, C₃₋₆cycloalkylC₁₋₄alkyl, C₁₋₄alkoxy, C₂₋₆alkenyl, hydroxyC₁₋₄alkyl, C₁₋₄alkoxyC₁₋₄alkyl, hydroxyC₁₋₄alkoxyC₁₋₄alkyl, cyanoC₁₋₄alkyl, haloC₁₋₄alkyl or aminocarboxyC₁₋₄alkyl;
R3 is H or C₁₋₄alkyl;
R4 and R5, which may be the same or different, are H, C₁₋₄alkyl, OH, halogen, C₁₋₄alkoxy, NR14R15 where each of R14 and R15, which may be the same or different, are H or C₁₋₄alkyl, NHCONR10R11 or OCONR10R11 where each of R10 and R11, which may be the same or different, are H or C₁₋₄alkyl or, together with the nitrogen to which they are attached, form a 5- or 6-membered heterocyclic group containing 0 to 3 further heteroatoms selected from N, O and S;
or R3 and R4 together with the interconnecting atoms form a 5- or 6-membered carbocyclic group or a heterocyclic group containing 1 heteroatom selected from N, O and S, which carbocyclic or heterocyclic group is optionally substituted with one group selected from C₁₋₄alkyl, OH, OC₁₋₄alkyl, halogen and NR16R17 where each of R16 and R17, which may be the same or different, are H or C₁₋₄alkyl;
R6 is H, C₁₋₄alkyl, halogen, OH, NHCO₂C₁₋₄alkyl, NR18R19 where each of R18 and R19, which may be the same or different, are H or C₁₋₄alkyl;
R7 is C₁₋₄alkyl;
or R4 and R7 together with the interconnecting atoms form a 5- to 7-membered heterocyclic group containing 0 or 1 further heteroatoms selected from N, O and S, which heterocyclic group is optionally substituted with one group selected from C₁₋₄₄alkyl, OH, OC₁₋₄alkyl, halogen and NR20R21 where each of R20 and R21, which may be the same or different, are H or C₁₋₄alkyl;
and
Het is an optionally substituted 4 to 7-membered non-aromatic heterocyclyl group containing 1 to 3 heteroatoms selected from N, O and S;
or a pharmaceutically acceptable salt thereof.

Examples of Het include pyrrolidinyl, pyrrolidin-2-on-yl, dioxolanyl, imidazolinyl, imidazolidin-2-on-yl, oxazolidinyl, oxazolidin-2-on-yl, pyrazolidinyl piperazinyl, ketopiperazinyl, diketopiperazinyl, piperidinyl, piperidin-2-on-yl, morpholinyl, thiomorpholinyl, morpholin-2-on-yl or isothiazolidinyl.

The Het group may be attached to the imidazopyridine core through either a carbon atom or a heteroatom of the Het ring. For example if the Het group is an pyrrolidinone group, the bond to the imidazopyridine core may be through either a carbon atom (C-linked) or a nitrogen atom (N-linked) on the pyrrolidinone Het group.

Optional substituents for the carbocyclyl and heterocyclyl groups are selected from halogen, hydroxy, oxo, cyano, nitro, (C₁₋₄)alkyl, (C₁₋₄)alkoxy, hydroxy(C₁₋₄)alkyl, hydroxy(C₁₋₄)alkoxy, halo(C₁₋₄)alkyl, halo(C₁₋₄)alkoxy, aryl(C₁₋₄)alkoxy, (C₁₋₄) alkylthio, (C₁₋₄alkoxy(C₁₋₄)alkyl, (C₃₋₆)cycloalkyl(C₁₋₄)alkoxy, (C₁₋₄)alkanoyl, (C₁₋₄alkylsulfonyl, (C₁₋₄)alkylsulfonyloxy, (C₁₋₄alkylsulfonyl(C₁₋₄alkyl, arylsulfonyl, arylsulfonyloxy, aryloxysulfonyl, arylsulfonyl(C₁₋₄)alkyl, (C₁₋₄)alkylsulfonamido(C₁₋₄) alkyl, (C₁₋₄)alkylamido(C₁₋₄)alkyl, arylsulfonamido, arylcarboxamido, arylsulfonamido(C₁₋₄)alkyl, arylcarboxamido(C₁₋₄alkyl, arylaminosulfonyl, arylaminocarbonyl, aroyl, aroyl(C₁₋₄)alkyl, or aryl(C₁₋₄)alkanoyl group; a group NR22R23, CO₂R22, CONR22R23, where each of R22 and R23, which may be the same or different, are H, C₁₋₄alkyl, hydroxyalkyl, alkoxyalkyl, cycloalkyl, cycloalkylalkyl, or together form an optionally substituted 5- or 6-membered heterocyclic group containing a further heteroatom selected from N, O or S.

When the compound contains an alkyl group, whether alone or forming part of a larger group, e.g. alkoxy or alkylthio, the alkyl group may be straight chain branched or cyclic, or combinations thereof.

In one embodiment Het is pyrrolidinon-yl, imidazolidinyl, piperidin-2 -on-yl, imidazolidin-2-on-yl, morpholin-2-on-yl, or hydroxy-2-pyrrolidinon-yl.

In a further embodiment Het is piperidin-2-on-yl or imidazolidinonyl.

In one embodiment the optional substituents on the Het group are (C₁₋₄)alkyl or hydroxy.

In a further embodiment (C₁₋₄)alkyl as optional substituents on the Het group are methyl or ethyl.

In one embodiment X is NH or O.

In a further embodiment X is NH.

In a further embodiment X is O.

In one embodiment R1 and R2 are both methyl.

In one embodiment R3 is H.

In one embodiment R4 and R5 are not both H.

In a further embodiment R4 and R5 are both methyl, or R4 is methyl and R5 is ethyl.

In one embodiment R6 is H.

In one embodiment X is NH, R1 and R2 are both methyl, R3 is H, R4 and R5 are both methyl and R6 is H.

In one embodiment X is O, R1 and R2 are both methyl, R3 is H, R4 and R5 are both methyl and R6 is H.

When used herein, halogens include fluoro, chloro, bromo and iodo.

When used herein the term aryl means a 5- to 6-membered aromatic ring for example phenyl, or a 7 to 12 membered bicyclic ring system where at least one of the rings is aromatic for example naphthyl.

It is to be understood that the present invention covers all combinations of particularised groups and substituents described herein above.

It will be appreciated that compounds of formula (I) may exist as R or S enantiomers. The present invention includes within its scope all such isomers, including mixtures. Where additional chiral centres are present in compounds of formula (I), the present invention includes within its scope all possible diastereoisomers, including mixtures thereof. The different isomeric forms may be separated or resolved one from the other by conventional methods, or any given isomer may be obtained by conventional synthetic methods or by stereospecific or asymmetric syntheses. The invention also extends to any tautomeric forms and mixtures thereof.

It will be understood that the invention includes pharmaceutically acceptable derivatives of compounds of formula (I) and that these are included within the scope of the invention.

Particular compounds according to the invention include those mentioned in the examples and their pharmaceutically acceptable derivatives.

As used herein “pharmaceutically acceptable derivative” includes any pharmaceutically acceptable salt, ester or salt of such ester of a compound of formula (I) which, upon administration to the recipient is capable of providing (directly or indirectly) a compound of formula (I) or an active metabolite or residue thereof.

It will be appreciated that for use in medicine the salts of the compounds of formula (I) should be pharmaceutically acceptable. Suitable pharmaceutically acceptable salts will be apparent to those skilled in the art. Pharmaceutically acceptable salts include those described by Berge, Bighley and Monkhouse, J. Pharm. Sci. (1977) 66, pp 1-19. Such pharmaceutically acceptable salts include acid addition salts formed with inorganic acids e.g. hydrochloric, hydrobromic, sulphuric, nitric or phosphoric acid; and organic acids e.g. succinic, maleic, acetic, fumaric, citric, tartaric, benzoic, p-toluenesulfonic, methanesulfonic or naphthalenesulfonic acid. Other salts e.g. oxalates or formates, may be used, for example in the isolation of compounds of formula (I) and are included within the scope of this invention. Also included within the scope of the invention are solvates and hydrates of compounds of formula (I).

Certain of the compounds of formula (I) may form acid addition salts with one or more equivalents of the acid. The present invention includes within its scope all possible stoichiometric and non-stoichiometric forms.

The compounds of formula (I) may be prepared in crystalline or non-crystalline form and, if crystalline, may optionally be solvated, eg. as the hydrate. This invention includes within its scope stoichiometric solvates (eg. hydrates) as well as compounds containing variable amounts of solvent (eg. water).

The subject invention also includes isotopically-labeled compounds which are identical to those recited in formula (I) and following, but for the fact that one or more atoms are replaced by an atom having an atomic mass or mass number different from the atomic mass or mass number most commonly found in nature. Examples of isotopes that can be incorporated into compounds of the invention include isotopes of hydrogen, carbon, nitrogen, oxygen, fluorine, iodine and chlorine such as 3H, 11C, 14C, 18F, 123I, 125I.

Compounds of the present invention and pharmaceutically acceptable salts of said compounds that contain the aforementioned isotopes and/or other isotopes of other atoms are within the scope of the present invention. Isotopically-labeled compounds of the present invention, for example those into which radioactive isotopes such as 3H or 14C have been incorporated, are useful in drug and/or substrate tissue distribution assays. Tritiated, ie. 3H, and carbon-14, ie. 14C, isotopes are particularly preferred for their ease of preparation and detectability. 11C and 18 F isotopes are particularly useful in PET (positron emission tomography).

Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

According to a further aspect of the present invention there is provided a process for the preparation of compounds of formula (I) and derivatives thereof. The following schemes detail some synthetic routes to compounds of the invention. In the following schemes reactive groups can be protected with protecting groups and deprotected according to well established techniques.

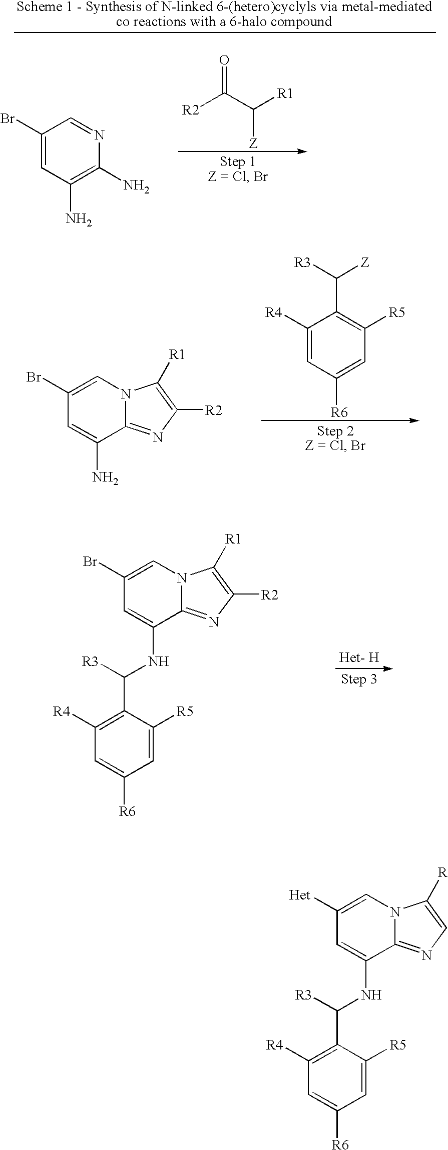

Thus, compounds of the invention for which X is NH can be synthesised as outlined in Scheme 1. Step 1 typically comprises reacting a diamino-halopyridine derivative with the appropriate haloketone in an appropriate solvent such as N-methylpyrrolidinone (NMP) under microwave conditions at an appropriate temperature such as 180° C. for an appropriate time such as 1 h. Alternatively, step 1 can be effected by heating at reflux in ethanol, or by heating at a suitable temperature in NMP. Step 2 consists of reacting the 8-amino-6-haloimidazopyridine with an appropriate benzyl halide such as the benzyl chloride in the presence of a base such as sodium carbonate in a suitable solvent such as dimethylformamide (DMF) for a suitable time such as 3-16 h. Additives such as potassium iodide may be used. In step 3, an appropriate metal-mediated coupling of a heterocyclyl group can be used. For example, Ullman-type couplings can be used, in which the 6-halo compound can be reacted in the presence of copper (I) iodide and a base such as potassium carbonate in a suitable solvent such as dioxane at a suitable temperature such as reflux for a suitable time such as 3 days. Alternatively, the reaction can be conducted under microwave conditions in a suitable solvent such as DMF or NMP at suitable temperatures up to 195° C. Additives such as trans 1,2-diaminocyclohexane may be used, and the base can alternatively be potassium phosphate. Alternatively the coupling may be performed in the presence of an appropriate palladium catalyst such as tris(dibenzylideneacetone)dipalladium(0) and a phosphine ligand such as 4,5-bis(diphenylphosphino)-9,9-dimethyl-xanthene, in the presence of a suitable base such as cesium carbonate in a suitable solvent system such as dioxane at a suitable temperature such as reflux for a suitable time such as 5 hours. This reaction, also, may alternatively be conducted under microwave conditions.

Alternatively, compounds of the invention wherein X is NH can be generated according to Scheme 2:

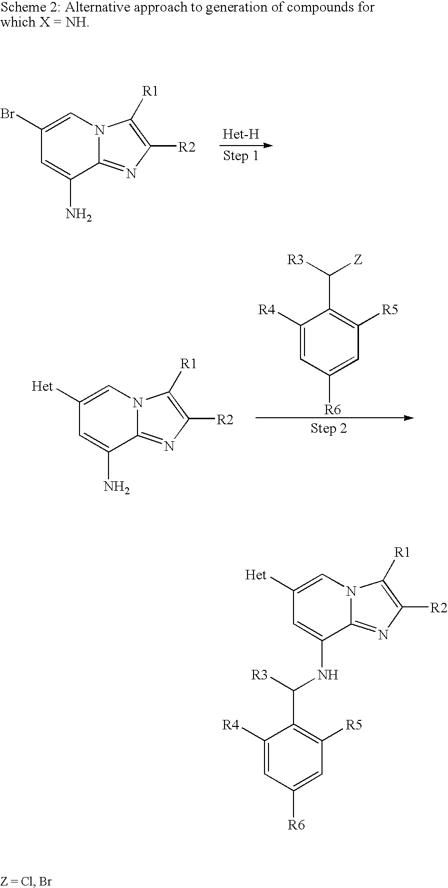

Typically, In step 1, an appropriate metal-mediated coupling of a heterocyclyl group can be used. For example, Ullman-type couplings can be used, in which the 6-halo compound can be reacted in the presence of copper (I) iodide and a base such as potassium carbonate in a suitable solvent such as dioxane at a suitable temperature such as reflux for a suitable time such as 3 days. Alternatively, the reaction can be conducted under microwave conditions in a suitable solvent such as DMF or NMP at suitable temperatures up to 195° C. Additives such as trans 1,2-diaminocyclohexane may be used, and the base can alternatively be potassium phosphate. Alternatively the coupling may be performed in the presence of an appropriate palladium catalyst such as tris(dibenzylideneacetone)dipalladium(0) and a phosphine ligand such as 4,5-bis(diphenylphosphino)-9,9-dimethyl-xanthene, in the presence of a suitable base such as cesium carbonate in a suitable solvent system such as dioxane at a suitable temperature such as reflux for a suitable time such as 5 hours. This reaction, also, may alternatively be conducted under microwave conditions.

Step 2 typically consists of reacting the product of step 1 with an appropriate benzyl halide such as the benzyl bromide in the presence of a base such as sodium carbonate in a suitable solvent such as DMF for a suitable time such as 3 h. Additives such as potassium iodide may be used.

Compounds of the invention for which X is O can be synthesised according to steps 1 to 3 of scheme 3:

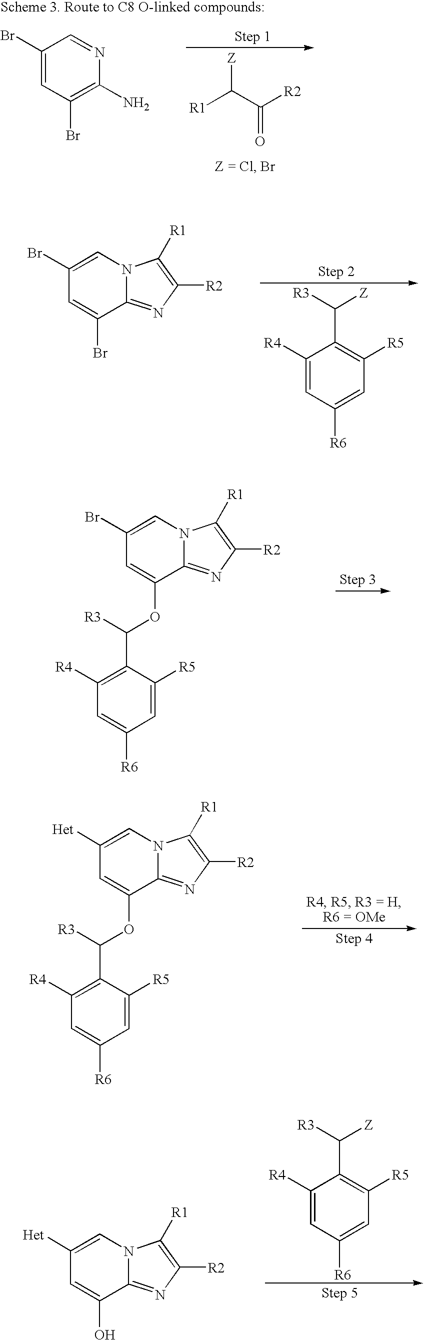

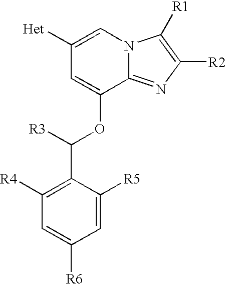

Step 1 typically comprises the use of an appropriate ketone such as alpha-chloro or alpha-bromo ketone, in the presence of a suitable solvent such as NMP, at a suitable temperature such as between 160° C. and 180° C. in the presence of microwaves. Step 2 typically comprises the use of the appropriate benzylic alkoxide (generated by the use of an appropriate base such as sodium hydride, in the presence of a suitable solvent such as DMF at a suitable temperature such as 0° C. to room temperature) in the presence of a suitable solvent such as DMF at an appropriate temperature such as between 60° C. and 90° C. Step 3 typically comprises the use of the appropriate heterocyclyl derivative in the presence of an appropriate catalyst such as copper (I) iodide and a base such as potassium carbonate in the presence of a suitable solvent such as NMP or DMF at a suitable temperature such as between 150° C. and 190° C. in the presence of microwaves.

Alternatively, compounds of the invention for which X is O can be generated according to steps 4 and 5 of scheme 3. Thus, when the groups R4, R5, R3 or R6 facilitate removal of the O-benzyl group, for example when R4, R5, R3 are H and R6 (in this scheme) is OMe, the product of step 3 can be subjected to steps 4 and 5 to provide alternative C8-modified analogues. Typically step 4 consists of treating with an acid such as trifluoroacetic acid in a suitable solvent such as dichloromethane, at a suitable temperature such as room temperature. Typically step 5 consists of treating with a suitable base such as sodium hydride in a suitable solvent such as DMF, followed by reacting with the appropriate benzylhalide, such as a benzylbromide, at a suitable temperature such as room temperature for an appropriate time, such as 16H.

It will be understood by those skilled in the art that certain compounds of the invention can be converted into other compounds of the invention according to standard chemical methods.

The starting materials for use in Schemes 1 to 3 are commercially available known in the literature or can be prepared by known methods.

The compounds of formula (I) may be prepared singly or as compound libraries comprising at least 2, e.g. 5 to 1000, preferably 10 to 100 compounds of formula (I). Compound libraries may be prepared by a combinatorial ‘split and mix’ approach or by multiple parallel synthesis using either solution phase or solid phase chemistry, by procedures known to those skilled in the art.

Thus according to a further aspect of the invention there is provided a compound library comprising at least 2 compounds of formula (I), or pharmaceutically acceptable derivatives thereof.

Pharmaceutically acceptable salts may be prepared conventionally by reaction with the appropriate acid or acid derivative.

The compounds of formula (I) and their pharmaceutically acceptable derivatives are useful for the treatment of diseases or disorders where an acid pump antagonist (APA) is required such as gastrointestinal diseases or disorders, for example those associated with hyperacidity. The compounds of the invention may be particularly useful for the treatment or prophylaxis of inflammatory gastrointestinal diseases and diseases associated with an imbalance in gastric acid such as gastric or duodenal ulcer, gastritis, gastro-oesophageal reflux disease (GERD), and Zoller-Ellison Syndrome or diseases and disorders where gastric antisecretory effect is desirable for example in patients with gastrinomas and acute upper gastrointestinal bleeding.

The invention also provides a method of treating or preventing diseases or disorders where an antagonist of a human acid pump is required, for example those diseases and disorders mentioned hereinabove, which comprises administering to a subject in need thereof an effective amount of a compound of formula (I), or a pharmaceutically acceptable derivative thereof.

The invention also provides a compound of formula (I), or a pharmaceutically acceptable derivative thereof, for use in the treatment or prophylaxis of diseases or disorders where an antagonist of a human acid pump is required, for example those diseases and disorders mentioned hereinabove.

The invention also provides the use of a compound of formula (I), or pharmaceutically acceptable derivative thereof, in the manufacture of a medicament for the treatment or prophylaxis of diseases or disorders where an antagonist of a human acid pump is required, for example those diseases and disorders mentioned hereinabove.

The invention also provides the use of a compound of formula (I), or a pharmaceutically acceptable derivative thereof, in the manufacture of a medicament for the treatment or prophylaxis of diseases or disorders where an antagonist of a human acid pump is required such as inflammatory gastrointestinal diseases and diseases associated with an imbalance in gastric acid such as gastric or duodenal ulcer, gastritis, gastro-oesophageal reflux disease (GERD), and Zoller-Ellison Syndrome or diseases and disorders where gastric antisecretory effect is desirable for example in patients with gastrinomas and acute upper gastrointestinal bleeding.

For use in therapy the compounds of the invention are usually administered as a pharmaceutical composition. The invention also provides a pharmaceutical composition comprising a compound of formula (I), or a pharmaceutically acceptable derivative thereof, and a pharmaceutically acceptable carrier.

The compounds of formula (I) and their pharmaceutically acceptable derivatives may be administered by any convenient method, e.g. by oral, parenteral, buccal, sublingual, nasal, rectal or transdermal administration, and the pharmaceutical compositions adapted accordingly.

The compounds of formula (I) and their pharmaceutically acceptable derivatives which are active when given orally can be formulated as liquids or solids, e.g. as syrups, suspensions, emulsions, tablets, capsules or lozenges.

A liquid formulation will generally consist of a suspension or solution of the active ingredient in a suitable liquid carrier(s) e.g. an aqueous solvent such as water, ethanol or glycerine, or a non-aqueous solvent, such as polyethylene glycol or an oil.

The formulation may also contain a suspending agent, preservative, flavouring and/or colouring agent.

A composition in the form of a tablet can be prepared using any suitable pharmaceutical carrier(s) routinely used for preparing solid formulations, such as magnesium stearate, starch, lactose, sucrose and cellulose.

A composition in the form of a capsule can be prepared using routine encapsulation procedures, e.g. pellets containing the active ingredient can be prepared using standard carriers and then filled into a hard gelatin capsule; alternatively a dispersion or suspension can be prepared using any suitable pharmaceutical carrier(s), e.g. aqueous gums, celluloses, silicates or oils and the dispersion or suspension then filled into a soft gelatin capsule.

Typical parenteral compositions consist of a solution or suspension of the active ingredient in a sterile aqueous carrier or parenterally acceptable oil, e.g. polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the active ingredient in a pharmaceutically acceptable aqueous or non-aqueous solvent and are usually presented in single or multidose quantities in sterile form in a sealed container which can take the form of a cartridge or refill for use with an atomising device. Alternatively the sealed container may be a disposable dispensing device such as a single dose nasal inhaler or an aerosol dispenser fitted with a metering valve. Where the dosage form comprises an aerosol dispenser, it will contain a propellant which can be a compressed gas e.g. air, or an organic propellant such as a fluorochlorohydrocarbon or hydrofluorocarbon. Aerosol dosage forms can also take the form of pump-atomisers.

Compositions suitable for buccal or sublingual administration include tablets, lozenges and pastilles where the active ingredient is formulated with a carrier such as sugar and acacia, tragacanth, or gelatin and glycerin.

Compositions for rectal administration are conveniently in the form of suppositories containing a conventional suppository base such as cocoa butter.

Compositions suitable for transdermal administration include ointments, gels and patches.

Preferably the composition is in unit dose form such as a tablet, capsule or ampoule.

The dose of the compound of formula (I), or a pharmaceutically acceptable derivative thereof, used in the treatment or prophylaxis of the above-mentioned disorders or diseases will vary in the usual way with the particular disorder or disease being treated, the weight of the subject and other similar factors. However, as a general rule, suitable unit doses may be 0.05 to 1000 mg, more suitably 0.05 to 500 mg. Unit doses may be administered more than once a day for example two or three times a day, so that the total daily dosage is in the range of about 0.01 to 100 mg/kg; and such therapy may extend for a number of weeks or months. In the case of pharmaceutically acceptable derivatives the above figures are calculated as the parent compound of formula (I).

No toxicological effects are indicated/expected when a compound of formula (I) is administered in the above mentioned dosage range.

All publications, including but not limited to patents and patent applications, cited in this specification are herein incorporated by reference as if each individual publication were specifically and individually indicated to be incorporated by reference herein as though fully set forth.

The following Examples illustrate the preparation of pharmacological active compounds of the invention.

### DESCRIPTION 1

### 1,1-Dimethylethyl 4-(8-{[(2,6-dimethylphenyl)methyl]amino}-2,3-dimethylimidazo[1,2-α]pyridin-6-yl)-1-piperazinecarboxylate

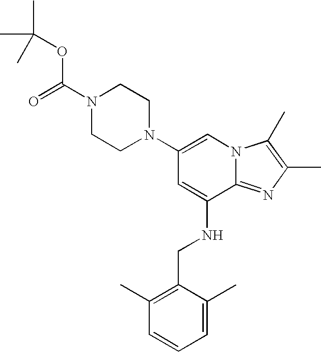

A mixture of 6-bromo-N-[(2,6-dimethylphenyl)methyl]-2,3-dimethylimidazo[1,2-α]pyridin-8-amine (200 mg, 0.558 mmol; WO 98/37080), 1,1-dimethylethyl 1-piperazinecarboxylate (153 mg, 0.84 mmol), 2-dicyclohexylphosphino-2′-(N,N-dimethylamino)biphenyl (33 mg, 0.083 mmol),

tris(dibenzylideneacetone)dipalladium(0) (25 mg, 0.028 mmol) and sodium tert-butoxide (107 mg, 1.12 mmol) in dioxane (4 mL) was heated in an Initiator™ Microwave Synthesizer at 120° C. for 1 hour. The cooled mixture was partitioned between ethyl acetate and water. The organic phase was washed with water, then brine, dried (MgSO₄) and evaporated. Purification by chromatography on silica gel (ethyl acetate/hexane) gave the title compound as a yellow gum. MS (ES+ve): [M+H]⁺ at m/z 464 (C₂₇H₃₇N₅O₂ requires [M+H]⁺ at m/z 464).

### DESCRIPTION 2

### 1-(8-Amino-2,3-dimethylimidazo[1,2-α]pyridin-6-yl)-2(1H)-pyridinone

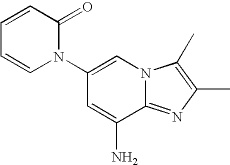

A mixture containing 6-bromo-2,3-dimethylimidazo[1,2-α]pyridin-8-amine (6 g, 25 mmol; WO 98/37080), 2-hydroxypyridine (4.75 g, 50 mmol), potassium carbonate (6.9 g, 50 mmol), copper (I) iodide (0.95 g, 5 mmol) and N-methylpyrrolidinone (50 mL) was stirred at 195° C. for 8 hours. The mixture was filtered through celite, washing with ethyl acetate and water, and then immobilised on SCX resin which were washed with methanol. The mixture was eluted with 2N NH₃/MeOH and solvent removed in vacuo. The mixture was purified on silica eluting with a 0-20% methanol in ethyl acetate gradient to give, upon evaporation, the title compound as a brown solid. 2.4 g. MS (ES+ve): [M+H]⁺ at m/z 255 (C₁₄H₁₄N₄O requires [M+H]⁺ at m/z 255).

### DESCRIPTION 3

### 1-(8-Amino-2,3-dimethylimidazo[1,2-α]pyridin-6-yl)-2-piperidinone

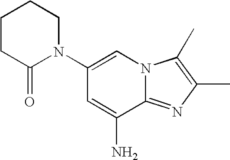

10% Palladium on carbon (10 mg) was added to a solution of 1-(8-amino-2,3-dimethylimidazo[1,2-α]pyridin-6-yl)-2(1H)-pyridinone (90.5 mg, 0.356 mmol; Description 2) in ethanol (20 mL), then the mixture was shaken under hydrogen (50 psi) for 16 hours. The mixture was filtered through a celite pad and the solvent evaporated to give the title compound. MS (ES+ve): [M+H]⁺ at m/z 259 (C₁₄H₁₈N₄O requires [M+H]⁺ at m/z 259).

### DESCRIPTION 4

### 6,8-Dibromo-2,3-dimethylimidazo[1,2-α]pyridine

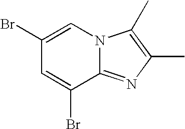

A mixture of 2-amino-3,5-dibromopyridine (5.45 g, 21.6 mmol), 3-bromo-2-butanone (3.64 g, 24.1 mmol) and N-methylpyrrolidinone (11 mL) was heated in an Initiator™ Microwave Synthesizer at 180° C. for 1 hour. After cooling, methanol (10 mL) was added and the mixture poured into ethyl acetate and aqueous sodium bicarbonate. The mixture was filtered, the filtrate layers were separated and the aqueous layer extracted with ethyl acetate. The combined organic extracts were washed twice with water, then brine, dried (MgSO₄) and evaporated. Purification by chromatography on silica gel (ethyl acetate/toluene) gave the title compound. MS (ES+ve): [M+H]⁺ at m/z 303 (C₉H₈⁷⁹Br₂N₂ requires [M+H]⁺ at m/z 303).

### DESCRIPTION 5

### 6-Bromo-2,3-dimethyl-8-({[4-(methyloxy)phenyl]methyl}oxy)imidazo[1,2-α]pyridine

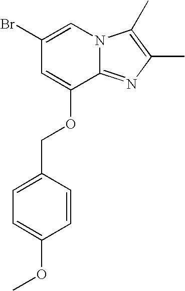

Sodium hydride (80 mg of a 60% dispersion in mineral oil; 2.0 mmol) was added to a stirring solution of [4-(methyloxy)phenyl]methanol (250 μL; 2.0 mmol) in dry dimethylformamide (3 mL) and the mixture stirred under argon for 90 minutes. 6,8-Dibromo-2,3-dimethylimidazo[1,2-α]pyridine (300 mg, 1.0 mmol; Description 4) was added and the resulting mixture was heated at 90° C. under argon for 18 hours. The mixture was applied to an Isolute® SCX cartridge and eluted with methanol followed by 2M NH₃ in methanol. The basic fractions were combined and evaporated under reduced pressure. The residue was purified by column chromatography on silica eluting with 2-1 n-pentane-ethyl acetate and then ethyl acetate to afford the title compound as a pale yellow solid (200 mg). ¹H NMR (CDCl₃) δ 2.35 (3H, s), 2.40 (3H, s), 3.81 (3H, s), 5.22 (2H, s), 6.54 (H, s), 6.90 (2H, m), 7.41 (2H, m), 7.57 (H, s)

### DESCRIPTION 6

### 1-[2,3-Dimethyl-8-({[4-(methyloxy)phenyl]methyl}oxy)imidazo[1,2-α]pyridin-6 -yl]-2(1H)-pyridinone

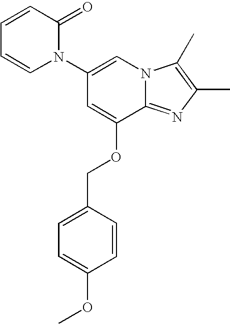

A mixture of 6-bromo-2,3-dimethyl-8-({[4-(methyloxy)phenyl]methyl}oxy)imidazo[1,2-α]pyridine (90 mg, 0.25 mmol. Description 5), 2(1H)-pyridinone (50 mg, 0.5 mmol), potassium carbonate (69 mg, 0.5 mmol) and copper (I) iodide (10 mg, 0.05 mmol) in dry dimethylformamide (3 mL) was heated in an Initiator™ Microwave Synthesizer at 150° C. for 4 hours. The mixture was filtered through Celite and the pad washed with ethyl acetate. The filtrate was evaporated under reduced pressure and the residue applied to an Isolute® SCX cartridge and eluted with methanol followed by 2M NH₃ in methanol. The basic fractions were combined and evaporated under reduced pressure. This residue was purified by column chromatography on silica eluting with ethyl acetate and then 3-97 2M NH₃ in methanol-dichloromethane to afford the title compound as a pale yellow gum (52 mg) MS (ES+ve): [M+H]⁺ at m/z 376 (C₂₂H₂₁N₃O₃ requires [M+H]⁺ at m/z 376).

### DESCRIPTION 7

### 1-(8-Hydroxy-2,3-dimethylimidazo[1,2-α]pyridin-6-yl)-2(1H)-pyridinone

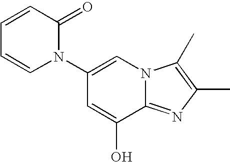

Trifluoroacetic acid (5 mL) was added to solution of 1-[2,3-dimethyl-8-({[4-(methyloxy)phenyl]methyl}oxy)imidazo[1,2-α]pyridin-6-yl]-2 (1H)-pyridinone (447 mg, 1.19 mmol; Description 6) in dichloromethane (5 mL) and the mixture stirred at room temperature for 3 hours. The mixture was purified on an Isolute® SCX cartridge and eluted with methanol followed by 2M NH₃ in methanol. The basic fractions were combined and evaporated under reduced pressure. This residue was triturated with diethyl ether and the title compound obtained by filtration as a buff powder (253 mg) MS (ES+ve): [M+H]⁺ at m/z 256 (C₁₄H₁₃N₃O₂ requires [M+H]⁺ at m/z 256).

### DESCRIPTION 8

### 1-{2,3-Dimethyl-8-[(3-methyl-2-buten-1-yl)oxy]imidazo[1,2-α]pyridin-6-yl}-2(1H)-pyridinone

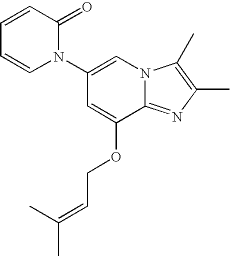

Sodium hydride (33 mg of a 60% dispersion in mineral iol, 0.82 mmol) was added to a solution of 1-(8-hydroxy-2,3-dimethylimidazo[1,2-α]pyridin-6-yl)-2(1H)-pyridinone (200 mg, 0.78 mmol; Description 7) in dimethylformamide (4 mL) and the mixture stirred for 1 hour. 4-Bromo-2-methyl-2-butene (95 Ξl, 0.82 mmol) was added and the mixture allowed to react at room temperature for 16 hours. The mixture was applied to an Isolute® SCX cartridge and eluted with methanol followed by 2M NH₃ in methanol. The basic fractions were combined and evaporated under reduced pressure. This residue was purified by column chromatography on silica eluting with 3-97 2M NH₃ in methanol-dichloromethane to afford the title compound as a colourless solid (87 mg) MS (ES+ve): [M+H]⁺ at m/z 324 (C₁₉H₂₁N₃O₂ requires [M+H]⁺ at m/z 324).

### DESCRIPTION 9

### 1-(8-Hydroxy-2,3-dimethylimidazo[1,2-α]pyridin-6-yl)-2-piperidinone

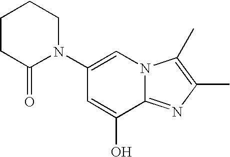

A solution of 1-{2,3-dimethyl-8-[(3-methyl-2-buten-1-yl)oxy]imidazo[1,2-α]pyridin-6-yl}-2(1H)-pyridinone (67 mg, 0.21 mmol, Description 8) in ethanol (3 mL) was hydrogenated at room temperature and pressure over a 50% wet paste of 10% palladium on carbon (10 mg) for 18 hours. The catalyst was removed by filtration through celite and the filtrate evaporated under reduced pressure to afford the title compound as a colourless powder (55 mg) MS (ES+ve): [M+H]⁺ at m/z 260 (C₁₄H₁₇N₃O₂ requires [M+H]⁺ at m/z 260).

### DESCRIPTION 10

### (4S)-1-[2,3-Dimethyl-8-({[4-(methyloxy)phenyl]methyl}oxy)imidazo[1,2-α]pyridin-6-yl]-4-hydroxy-2-pyrrolidinone

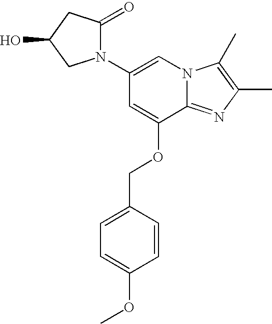

A mixture of 6-bromo-2,3-dimethyl-8-({[4-(methyloxy)phenyl]methyl}oxy)imidazo[1,2-α]pyridine (500 mg, 1.38 mmol. Description 5), (5) 4-hydroxy-pyrrolidin-2-one (279 mg, 2.76 mmol), potassium carbonate (666 mg, 4.83 mmol) and copper(I) iodide (76 mg, 0.4 mmol) along with racemic-trans-N,N′-dimethylcyclohexane-1,2-diamine (56 mg, 0.4 mmol) in dry dimethylformamide (10 mL) was heated in an Initiator™ Microwave Synthesizer in 2 equal vessels at 120° C. for 4 hours. The cooled mixture was applied to an Isolute® SCX cartridge and washed with methanol followed by elution with 2M NH₃ in methanol. The basic fractions were combined and evaporated under reduced pressure. This residue was purified by column chromatography on silica eluting with a 0-10% methanol in ethyl acetate gradient to afford the title compound MS (ES+ve): [M+H]⁺ at m/z 382 (C₂₁H₂₃N₃O₄ requires [M+H]⁺ at m/z 382)

### DESCRIPTION 11

### (4S)-4-Hydroxy-1-(8-hydroxy-2,3-dimethylimidazo[1,2-α]pyridin-6-yl)-2-pyrrolidinone

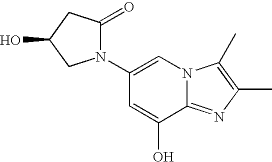

Trifluoroacetic acid (0.5 mL) was added to solution of (4S)-1-[2,3-dimethyl-8-({[4-(methyloxy)phenyl]methyl}oxy)imidazo[1,2-α]pyridin-6-yl]-4-hydroxy-2-pyrrolidinone (155 mg, 0.41 mmol; Description 10) in dichloromethane (10 mL) and the mixture stirred at room temperature for 15 minutes. The mixture was purified on an Isolute® SCX cartridge and eluted with methanol followed by 2M NH₃ in methanol. The basic fractions were combined and evaporated under reduced pressure to yield the title compound. MS (ES+ve): [M+H]⁺ at m/z 262 (C₁₃H₁₅N₃O₃ requires [M+H]⁺ at m/z 262).

### DESCRIPTION 12

### 4-[2,3-Dimethyl-8-({[4-(methyloxy)phenyl]methyl}oxy)imidazo[1,2-α]pyridin-6-yl]-3-morpholinone

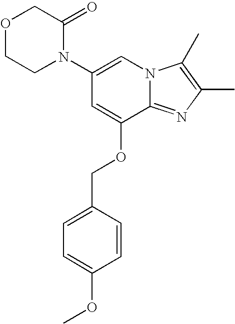

A mixture of 6-bromo-2,3-dimethyl-8-({[4-(methyloxy)phenyl]methyl}oxy)imidazo[1,2-α]pyridine (589 mg, 1.63 mmol; Description 5), 3-morpholinone (330 mg, 3.27 mmol; U.S. Pat. No. 3,308,121), cesium carbonate (1.86 g, 5.7 mmol), copper(I) iodide (89 mg, 0.47 mmol) and N,N′-dimethylethylenediamine (42 mg, 0.47 mmol) in N-methylpyrrolidinone (10 mL) was heated in an Initiator™ Microwave Synthesizer at 120° C. for 18 hours. The mixture was applied to an Isolute® SCX cartridge and washed with methanol followed by elution with 2M NH₃ in methanol. The basic fractions were combined and evaporated under reduced pressure. This residue was purified by column chromatography on silica eluting with a 50-100% ethyl acetate in hexane gradient to afford the title compound. MS (ES+ve): [M+H]⁺ at m/z 382 (C₂₁H₂₃N₃O₄ requires [M+H]⁺ at m/z 382):

### DESCRIPTION 13

### 4-(8-hydroxy-2,3-dimethylimidazo[1,2-α]pyridin-6-yl)-3-morpholinone

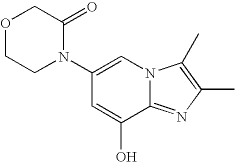

Trifluoroacetic acid (1.0 mL) was added to a solution of 4-[2,3-dimethyl-8-({[4-(methyloxy)phenyl]methyl}oxy)imidazo[1,2-α]pyridin-6-yl]-3-morpholinone (300 mg, 0.79 mmol; Description 12) in dichloromethane (15 mL) and the mixture stirred at room temperature for 105 minutes. The mixture was purified on an Isolute® SCX cartridge and washed with methanol followed by elution with 2M NH₃ in methanol. The basic fractions were combined and evaporated under reduced pressure to yield the title compound; MS (ES+ve): [M+H]⁺ at m/z 262 (C₁₃H₁₅N₃O₃ requires [M+H]⁺ at m/z 262).

### DESCRIPTION 14

### 1,1-Dimethylethyl (6-bromo-2,3-dimethylimidazo[1,2-α]pyridin-8-yl)carbamate

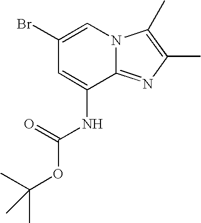

To a solution of 6-bromo-2,3-dimethylimidazo[1,2-α]pyridin-8-amine (450 mg, 1.88 mmol, WO 98/37080) in tetrahydrofuran (16 mL) was added sodium bis(trimethylsilyl)amide (4.3 mL, 4.30 mmol, 1.0M solution in tetrahydrofuran). The mixture was stirred at room temperature for 10 minutes and then a solution of di-tert-butyldicarbonate (490 mg, 2.24 mmol) in tetrahydrofuran (4 mL) was added. After stirring for 2 hours, the reaction mixture was quenched with aqueous ammonium chloride solution and the product extracted into ethyl acetate, dried (MgSO₄) and the solvent evaporated. The crude product was purified by chromatography on silica gel (ethyl acetate/hexane) to give the title compound.

### DESCRIPTION 15

### 1,1-Dimethylethyl[2,3-dimethyl-6-(2-oxo-1-pyrrolidinyl)imidazo[1,2-α]pyridin-8-yl]carbamate

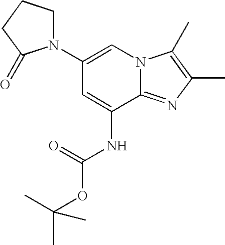

To a solution of 1,1-dimethylethyl (6-bromo-2,3-dimethylimidazo[1,2-α]pyridin-8-yl)carbamate (100 mg, 0.30 mmol; Description 14) in dioxane (4 mL) was added 2-pyrrolidinone (23 uL, 0.30 mmol), potassium carbonate (194 mg, 1.40 mmol), copper iodide (23 mg, 0.12 mmol) and N,N′-dimethylethylenediamine (13 uL, 0.12 mmol). The mixture was heated to reflux for 16 hours and then cooled to room temperature. The reaction was then diluted with ethyl acetate and water. After separation of the layers, the aqueous phase was re-extracted with ethyl acetate and then the combined organic layers were dried (MgSO₄) and the solvent evaporated. The crude product was purified by chromatography on silica gel (ethyl acetate/hexane) to give the title compound. MS (ES+ve): [M+H]⁺ at m/z 345 (C₁₈H₂₄N₄O₃ requires [M+H]⁺ at m/z 345).

### DESCRIPTION 16

### 1-(8-Amino-2,3-dimethylimidazo[1,2-α]pyridin-6-yl)-2-pyrrolidinone

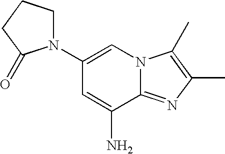

Trifluoroacetic acid (1 mL) was added to a solution of 1,1-dimethylethyl[2,3-dimethyl-6-(2-oxo-1-pyrrolidinyl)imidazo[1,2-α]pyridin-8-yl]carbamate (30 mg, 0.09 mmol; Description 15) in dichloromethane (3 mL) at 0° C. The reaction mixture was allowed to warm to room temperature and stirred for a further 4 hours. The mixture was diluted with dichloromethane and aqueous sodium hydrogen carbonate solution. After separation of the layers, the aqueous phase was further extracted with dichloromethane. The combined organic layers were dried (MgSO₄) and the solvent evaporated to give the title compound; MS (ES+ve): [M+H]⁺ at m/z 245 (C₁₃H₁₆N₄O requires [M+H]⁺ at m/z 245).

### EXAMPLE 1

### 1-(8-{[(2,6-Dimethylphenyl)methyl]amino}-2,3-dimethylimidazo[1,2-α]pyridin-6-yl)-2-pyrrolidinone hydrochloride

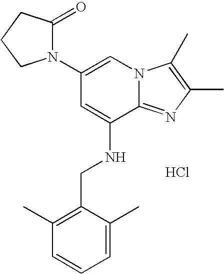

A mixture of 6-bromo-N-[(2,6-dimethylphenyl)methyl]-2,3-dimethylimidazo[1,2-α]pyridin-8-amine (50 mg, 0.140 mmol; WO 98/37080), 2-pyrrolidinone (24 mg, 0.279 mmol), tris(dibenzylideneacetone)dipalladium (6 mg, 0.007 mmol), 4,5-bis(diphenylphosphino)-9,9-dimethyl-xanthene (12 mg, 0.0209 mmol), cesium carbonate (68 mg, 0.209 mmol) and dioxane (2 mL) was heated under reflux under argon for 24 h. The cooled mixture was partitioned between ethyl acetate and water. The organic phase was washed with water, dried (MgSO₄) and evaporated. Purification by chromatography on silica gel (ethyl acetate/hexane) gave the free base of the title compound. This was dissolved in dichloromethane (2 mL), and then 1M HCl in diethyl ether (1 mL) was added. After stirring for 2 h, the solvents were evaporated. The residue was dissolved in water and freeze dried to give the title compound as a solid; MS (ES+ve): [M+H]⁺ at m/z 363 (C₂₄H₂₅N₃ requires [M+H]⁺ at m/z 363).

### EXAMPLE 2

### 1-(8-{[(2,6-dimethylphenyl)methyl]amino}-2,3-dimethylimidazo[1,2-α]pyridin-6-yl)-2-imidazolidinone hydrochloride

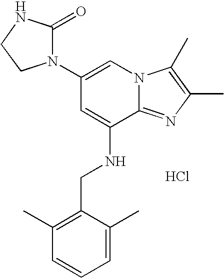

A mixture of 6-bromo-N-[(2,6-dimethylphenyl)methyl]-2,3-dimethylimidazo[1,2-α]pyridin-8-amine (400 mg, 1.117 mmol; WO 98/37080), 2-imidazolidinone (576 mg, 6.704 mmol), tris(dibenzylideneacetone) dipalladium (51 mg, 0.056 mmol), 4,5-bis(diphenylphosphino)-9,9-dimethyl-xanthene (97 mg, 0.168 mmol), cesium carbonate (545 mg, 1.676 mmol) and dioxane (24 mL) was heated under reflux under argon for 5 hours. The cooled mixture was partitioned between ethyl acetate and water. The organic phase was washed with water, dried (MgSO₄) and evaporated. Purification by chromatography on silica gel (ethyl acetate/hexane) gave the free base of the title compound which was triturated with diethyl ether. This was dissolved in dichloromethane (2 mL), and then 1M HCl in diethyl ether (1 mL) was added. After stirring for 3 hours, the solvents were evaporated to give the title compound as a yellow solid; MS (ES+ve): [M+H]⁺ at m/z 364 (C₂₄H₂₅N₃ requires [M+H]⁺ at m/z 364).

### EXAMPLE 3

### N-[(2,6-Dimethylphenyl)methyl]-2,3-dimethyl-6-(4-morpholinyl)imidazo[1,2-α]pyridin-8-amine hydrochloride

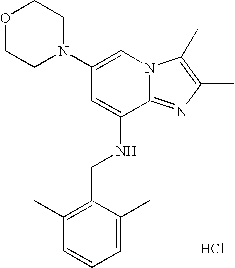

A mixture of 6-bromo-N-[(2,6-dimethylphenyl)methyl]-2,3-dimethylimidazo[1,2-α]pyridin-8-amine (100 mg, 0.279 mmol; WO 98/37080), morpholine (0.0486 mL, 0.556 mmol), 2-dicyclohexylphosphino-2′-(N,N-dimethylamino)biphenyl (16.7 mg, 0.042 mmol), tris(dibenzylideneacetone)dipalladium(0) (12.7 mg, 0.014 mmol) and sodium tert-butoxide (53.7 mg, 0.56 mmol) in dioxane (1.5 mL) was heated in an Initiator™ Microwave Synthesizer at 120° C. for 40 minutes. The cooled mixture was partitioned between ethyl acetate and water. The organic phase was washed with water, then brine, dried (MgSO₄) and evaporated. The residue was purified by chromatography on silica gel (ethyl acetate). The product was dissolved in methanol (3 mL), water (1 mL) and 2N HCl (0.3 mL) were added. After stirring for 5 minutes, the mixture was evaporated to give the title compound as a buff solid. MS (ES+ve): [M+H]⁺ at m/z 365 (C₂₂H₂₈N₄₀ requires [M+H]⁺ at m/z 365).

### EXAMPLE 4

### N-[(2,6-Dimethylphenyl)methyl]-2,3-dimethyl-6-(1-piperidinyl)imidazo[1,2-α]pyridin-8-amine hydrochloride

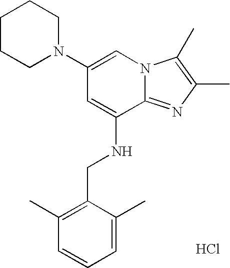

A mixture of 6-bromo-N-[(2,6-dimethylphenyl)methyl]-2,3-dimethylimidazo]1,2-α]pyridin-8-amine (100 mg, 0.279 mmol; WO 98/37080), piperidine (0.0553 mL, 0.56 mmol), 2-dicyclohexylphosphino-2′-(N,N-dimethylamino)biphenyl (16.7 mg, 0.042 mmol), tris(dibenzylideneacetone)dipalladium(0) (12.7 mg, 0.014 mmol) and sodium tert-butoxide (53.7 mg, 0.56 mmol) in dioxane (1.5 mL) was heated in an Initator™ Microwave Synthesizer at 120° C. for 1 hour. The cooled mixture was partitioned between ethyl acetate and water. The organic phase was washed with water, then brine, dried (MgSO₄) and evaporated. The residue was purified by chromatography on silica gel (ethyl acetate/hexane). The product was dissolved in methanol (3 mL), water (1 mL) and 2N HCl (0.3 mL) were added. After stirring for 5 minutes, the mixture was evaporated to give the title compound as a buff solid. MS (ES+ve): [M+H]⁺ at m/z 363 (C₂₃H₃₀N₄ requires [M+H]⁺ at m/z 363).

### EXAMPLE 5

### N-[(2,6-Dimethylphenyl)methyl]-2,3-dimethyl-6-(1-piperazinyl)imidazo[1,2-α]pyridin-8-amine hydrochloride

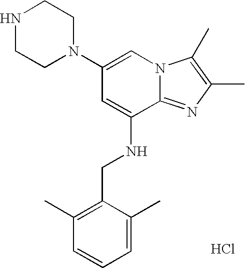

To a solution of 1,1-dimethylethyl 4-(8-{[(2,6-dimethylphenyl)methyl]amino}-2,3-dimethylimidazo[1,2-α]pyridin-6-yl)-1-piperazinecarboxylate (64 mg, 0.138 mmol; Description 1) in dichloromethane (15 mL), cooled in an ice bath, was added trifluoroacetic acid (0.5 mL). The mixture was stirred in the ice bath for 1 hour, then allowed to warm to room temperature and stirred for a further 2 hours. Toluene was added and the mixture was evaporated to dryness, a further portion of toluene was added and evaporated. The residue was dissolved in methanol and applied to an Isolute® SCX cartridge, elution with methanol, then 1M NH₃ in methanol gave, after evaporation, the product as the free base. This was dissolved in methanol/water/2N HCl (3:45:0.5 mL) and then loaded onto a 10 g tC18 Sep-Pak® cartridge and eluted with a gradient up to methanol/water/2N HCl (40:60:0.5) to give the title compound as a buff solid. MS (ES+ve): [M+H]⁺ at m/z 364 (C₂₂H₂₉N₅ requires [M+H]⁺ at m/z 364).

### EXAMPLE 6

### 1-(8-{[(2,6-Dimethylphenyl)methyl]amino}-2,3-dimethylimidazo[1,2-α]pyridin-6-yl)-2-piperidinone hydrochloride

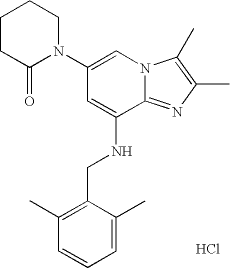

To a solution of 1-(8-amino-2,3-dimethylimidazo[1,2-α]pyridin-6-yl)-2-piperidinone (81.2 mg, 0.314 mmol; Description 3) in dimethylformamide (4 mL) was added sodium carbonate (83 mg, 0.783 mmol) and 2,6-dimethylbenzyl bromide (78 mg, 0.392 mmol). The mixture was stirred at room temperature for 20 hours, and then partitioned between ethyl acetate and water. The organic phase was washed with water, then brine, dried (MgSO₄) and evaporated. The residue was purified by chromatography on silica gel (ethyl acetate/methanol). The product was dissolved in methanol (3 mL), water (1 mL) and 2N HCl (0.2 mL) were added. After stirring for 5 minutes, the solution was evaporated to give the title compound as a buff solid. MS (ES+ve): [M+H]⁺ at m/z 377 (C₂₃H₂₈N₄O requires [M+H]⁺ at m/z 377).

### EXAMPLE 7

### 1-(8-{[(2,6-Dimethylphenyl)methyl]amino}-2,3-dimethylimidazo[1,2-α]pyridin-6-yl)-3-methyl-2-imidazolidinone hydrochloride

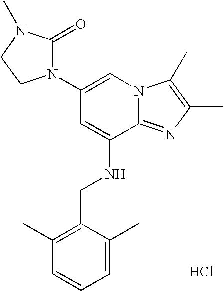

To a solution of 6-bromo-N-[(2,6-dimethylphenyl)methyl]-2,3-dimethylimidazo[1,2-α]pyridin-8-amine (370 mg, 1.03 mmol; WO 98/37080) in dioxane (22 mL) was added 1-methyl-2-imidazolidinone (620 mg, 6.20 mmol), cesium carbonate (504 mg, 1.55 mmol) and 4,5-bis(diphenylphosphino)-9,9-dimethyl-xanthene (90 mg, 0.16 mmol). After degassing the reaction mixture with argon, tris(dibenzylideneacetone)dipalladium (47 mg, 0.052 mmol) was added and the mixture heated to reflux for 5 hours. After cooling to room temperature, the dioxane was evaporated at reduced pressure. The residue was diluted with ethyl acetate, washed with water, dried (MgSO₄) and the solvent evaporated. The crude product was purified by chromatography on silica gel (ethyl acetate/hexane). The product was dissolved in dichloromethane (2 mL) and then 1 MHCl in diethyl ether (1 mL) was added. After stirring for 5 minutes, the solvents were evaporated to yield the title compound. MS (ES+ve): [M+H]⁺ at m/z 378 (C₂₂H₂₇N₅O requires [M+H]⁺ at m/z 378).

### EXAMPLE 8

### 1-(8-{[(2,6-Dimethylphenyl)methyl]oxy}-2,3-dimethylimidazo[1,2-α]pyridin-6-yl)-2-piperidinone

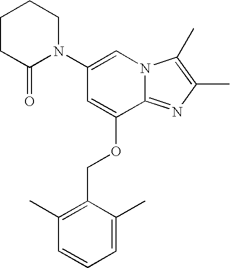

Sodium hydride (9 mg of a 60% dispersion in mineral iol, 0.21 mmol) was added to a solution of 1-(8-hydroxy-2,3-dimethylimidazo[1,2-α]pyridin-6-yl)-2(1H)-pyridinone (55 mg, 0.21 mmol; Description 9) in dimethylformamide (3 mL) and the mixture stirred for 1 hour. 2-(Chloromethyl)-1,3-dimethylbenzene (29 mg, 0.21 mmol) was added and the mixture allowed to react at room temperature for 18 hours. The mixture was applied to an Isolute® SCX cartridge and eluted with methanol followed by 2M NH₃ in methanol. The basic fractions were combined and evaporated under reduced pressure. This residue was purified by column chromatography on silica eluting with 3-97 2M NH₃ in methanol-dichloromethane to afford the title compound as a colourless powder; MS (ES+ve): [M+H]⁺ at m/z 378 (C₂₃H₂₇N₃O₂ requires [M+H]⁺ at m/z 378).

### EXAMPLE 9

### N-[(2,6-dimethylphenyl)methyl]-6-[cis-3,5-dimethyl-1-piperazinyl]-2,3-dimethylimidazo[1,2-α]pyridin-8-amine hydrochloride

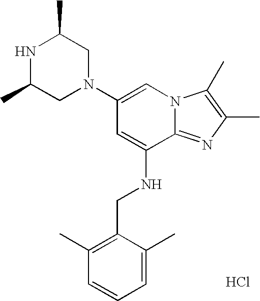

A mixture of 6-bromo-N-[(2,6-dimethylphenyl)methyl]-2,3-dimethylimidazo[1,2-α]pyridin-8-amine (30 mg, 0.0837 mmol; WO 98/37080), cis-2,6-dimethylpiperazine (14.3 mg, 0.126 mmol), 2-dicyclohexylphosphino-2′-(N,N-dimethylamino)biphenyl (5 mg, 0.0126 mmol), tris(dibenzylideneacetone)dipalladium(0) (3.8 mg, 0.0042 mmol) and sodium tert-butoxide (16.1 mg, 0.167 mmol) in dioxane (0.5 mL) was heated in an Initiator™ Microwave Synthesizer at 120° C. for 20 minutes. The reaction was repeated on a 2-fold scale, except that microwave heating was conducted for 30 minutes. The cooled reaction mixtures were combined and partitioned between ethyl acetate and dilute ammonium hydroxide. The aqueous phase was extracted with ethyl acetate and the combined organic phases were washed with water and then evaporated. The residue was dissolved in methanol/water/2N HCl (3:45:0.5 mL) and then loaded onto a 10 g tC18 Sep-Pak® cartridge and eluted with a gradient up to methanol/water/2N HCl (60:40:0.5) to give the title compound as a buff solid. MS (ES+ve): [M+H]⁺ at m/z 392 (C₂₄H₃₃N₅ requires [M+H]⁺ at m/z 392).

### EXAMPLE 10

### N-[(2,6-Dimethylphenyl)methyl]-6-(1,1-dioxido-2-isothiazolidinyl)-2,3-dimethylimidazo[1,2-α]pyridin-8-amine hydrochloride

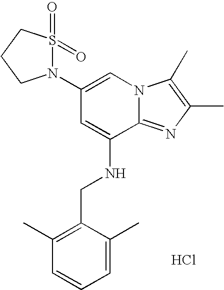

A mixture of 6-bromo-N-[(2,6-dimethylphenyl)methyl]-2,3-dimethylimidazo[1,2-α]pyridin-8-amine (100 mg, 0.28 mmol; WO 98/37080), isothiazolidine 1,1-dioxide (67 mg, 0.56 mmol; WO 04/050619), copper(I) iodide (16 mg, 0.083 mmol), potassium carbonate (138 mg, 1.0 mmol) and N,N′-dimethylethylenediamine (7.4 mg, 0.083 mmol) in dioxane (2 mL) was heated in an Initiator™ Microwave Synthesizer at 140° C. for 12 hours. The cooled mixture was applied to an Isolute® SCX cartridge. Elution with methanol, followed by water, then methanol then 1M NH₃ in methanol gave, after evaporation, the product which was further purified by chromatography on silica gel. Elution with dichloromethane/methanol (0 to 10%) gave a pale yellow solid which was dissolved in dichloromethane (2 mL), Ethereal HCl (1M; 1.0 mL) was added and the solvent evaporated. The residue was triturated under ether (1 mL) and filtered to give the title compound as a colourless solid; MS (ES+ve): [M+H]⁺ at m/z 399 (C₂₁H₂₆N₄O₂S requires [M+H]⁺ at m/z 399).

### EXAMPLE 11

### 4-(8-{[(2,6-Dimethylphenyl)methyl]amino}-2,3-dimethylimidazo[1,2-α]pyridin-6-yl)-3-morpholinone hydrochloride

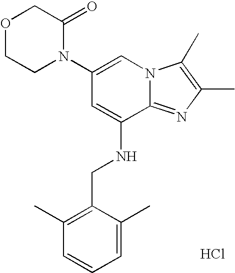

A mixture of 6-bromo-N-[(2,6-dimethylphenyl)methyl]-2,3-dimethylimidazo[1,2-α]pyridin-8-amine (100 mg, 0.28 mmol; WO 98/37080), 3-morpholinone (56 mg, 0.56 mmol; U.S. Pat. No. 3,308,121), copper(I) iodide (16 mg, 0.083 mmol), potassium carbonate (138 mg, 1.0 mmol) and N,N′-dimethylethylenediamine (7.4 mg, 0.083 mmol) in dioxane (2 mL) was heated in an Initiator™ Microwave Synthesizer at 140° C. for 12 hours. The cooled mixture was applied to an Isolute® SCX cartridge. Elution with methanol, followed by water, then methanol then 1M NH₃ in methanol gave, after evaporation, the product which was further purified by chromatography on silica gel. Elution with dichloromethane/methanol (0 to 10%) gave a pale yellow solid which was dissolved in methanol (2 mL), Ethereal HCl (1M; 0.3 ml) was added and the solvent evaporated to give the title compound as a pale yellow solid; MS (ES+ve):

[M+H]⁺ at m/z 379 (C₂₂H₂₆N₄O₂ requires [M+H]⁺ at m/z 379).

### EXAMPLE 12

### (4S)-1-(8-{[(2,6-dimethylphenyl)methyl]amino}-2,3-dimethylimidazo[1,2-α]pyridin-6-yl)-4-hydroxy-2-pyrrolidinone hydrochloride

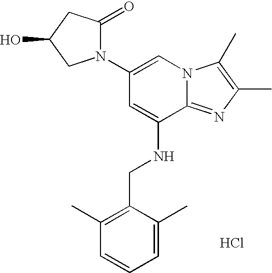

A mixture of 6-bromo-N-[(2,6-dimethylphenyl)methyl]-2,3-dimethylimidazo[1,2-α]pyridin-8-amine (200 mg, 0.558 mmol; WO 98/37080), (4S)-4-hydroxy-2-pyrrolidinone (112 mg, 1.12 mmol), copper(I) iodide (32 mg, 0.167 mmol), potassium carbonate (276 mg, 2.0 mmol) and N,N′-dimethylethylenediamine (15 mg, 0.167 mmol) in dioxane (2 mL) was heated in an Initiator™ Microwave Synthesizer at 140° C. for 14 hours. The cooled mixture was applied to an Isolute® SCX cartridge. Elution with methanol, followed by water, then methanol then 1M NH₃ in methanol gave, after evaporation, the product which was further purified by chromatography on silica gel. Elution with dichloromethane/methanol (0 to 10%) gave a pale yellow solid which was dissolved in methanol (2 mL), Ethereal HCl (1M; 0.5 ml) was added and the solvent evaporated to give the title compound as a brick-red coloured solid. MS (ES+ve): [M+H]⁺ at m/z 379 (C₂₂H₂₆N₄O₂ requires [M+H]⁺ at m/z 379).

### EXAMPLE 13

### (4R)-1-(8-{[(2,6-dimethylphenyl)methyl]amino}-2,3-dimethylimidazo[1,2-α]pyridin-6-yl)-4-hydroxy-2-pyrrolidinone hydrochloride

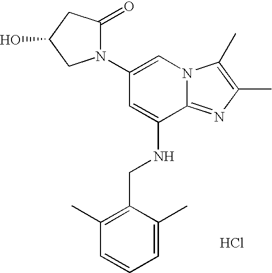

A mixture of 6-bromo-N-[(2,6-dimethylphenyl)methyl]-2,3-dimethylimidazo[1,2-α]pyridin-8-amine (200 mg, 0.558 mmol; WO 98/37080), (4R)-4-hydroxy-2-pyrrolidinone (112 mg, 1.12 mmol), copper(I) iodide (32 mg, 0.167 mmol), potassium carbonate (276 mg, 2.0 mmol) and N,N′-dimethylethylenediamine (15 mg, 0.167 mmol) in dioxane (2 mL) was heated in an Initiator™ Microwave Synthesizer at 140° C. for 14 hours. The cooled mixture was applied to an Isolute® SCX cartridge. Elution with methanol, followed by water, then methanol then 1M NH₃ in methanol gave, after evaporation, the product which was further purified by chromatography on silica gel. Elution with dichloromethane/methanol (0 to 10%) gave a pale yellow solid which was dissolved in methanol (2 mL), Ethereal HCl (1M; 0.5 ml) was added and the solvent evaporated to give the title compound as a brick-red coloured solid. MS (ES+ve): [M+H]⁺ at m/z 379 (C₂₂H₂₆N₄O₂ requires [M+H]⁺ at m/z 379).

### EXAMPLE 14

### (4S)-1-(8-{[(2,6-Dimethylphenyl)methyl]oxy}-2,3-dimethylimidazo[1,2-α]pyridin-6-yl)-4-hydroxy-2-pyrrolidinone hydrochloride

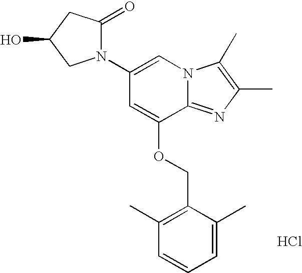

A mixture of potassium carbonate (52 mg, 0.38 mmol), (4S)-4-hydroxy-1-(8-hydroxy-2,3-dimethylimidazo[1,2-α]pyridin-6-yl)-2-pyrrolidinone (66 mg, 0.25 mmol; Description 11) and 2,6-dimethylbenzyl bromide (60 mg, 0.3 mmol) in dimethylformamide (2 mL) was stirred under argon at ambient temperature for 3 hours. The mixture was applied to an Isolute® SCX cartridge and eluted with methanol followed by 2M NH₃ in methanol. The basic fractions were combined and evaporated under reduced pressure. This residue was purified by column chromatography on silica eluting with a hexane to ethyl acetate to 10% methanol in ethyl acetate gradient to afford the free base of the title compound after evaporation under reduced pressure. This was dissolved in dichloromethane (2 mL), and then 1M HCl in diethyl ether (0.3 mL) was added. The solvents were evaporated to give the title compound; MS (ES+ve): [M+H]⁺ at m/z 380 (C₂₂H₂₅N₃O₃ requires [M+H]⁺ at m/z 380).

### EXAMPLE 15

### 4-(8-{[(2,6-dimethylphenyl)methyl]oxy}-2,3-dimethylimidazo[1,2-α]pyridin-6-yl)-3-morpholinone hydrochloride

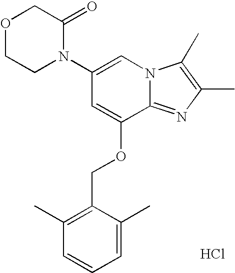

A mixture of potassium carbonate (143 mg, 1.04 mmol), 4-(8-hydroxy-2,3-dimethylimidazo[1,2-α]pyridin-6-yl)-3-morpholinone (180 mg, 0.69 mmol; Description 13) and 2,6-dimethylbenzyl bromide (165 mg, 0.83 mmol) in dimethylformamide (5 mL) was stirred under argon at ambient temperature overnight. The mixture was applied to an Isolute® SCX cartridge and washed with methanol followed by elution with 2M NH₃ in methanol. The basic fractions were combined and evaporated under reduced pressure. This residue was purified by column chromatography on silica eluting with a hexane to ethyl acetate gradient, evaporated under reduced pressure and dissolved in dichloromethane (2 mL). 1.0M ethereal HCl was added (0.5 mL) and the solution evaporated under reduced pressure to yield the title compound; MS (ES+ve): [M+H]⁺ at m/z 380 (C₂₂H₂₅N₃O₃ requires [M+H]⁺ at m/z 380).

### EXAMPLE 16

### 1-(8-{[(2-Ethyl-6-methylphenyl)methyl]amino}-2,3-dimethylimidazo[1,2-α]pyridin-6-yl)-2-pyrrolidinone hydrochloride

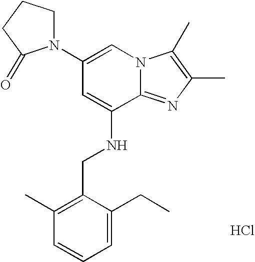

To a solution of 1-(8-amino-2,3-dimethylimidazo[1,2-α]pyridin-6-yl)-2-pyrrolidinone (14 mg, 0.057 mmol; Description 16) in dimethylformamide (2 mL) was added 2-(bromomethyl)-1-ethyl-3-methylbenzene (14 mg, 0.066 mmol) and sodium carbonate (22 mg, 0.208 mmol). The reaction mixture was stirred at room temperature for 16 hours and then treated with a further portion of 2-(bromomethyl)-1-ethyl-3-methylbenzene (8 mg, 0.038 mmol). The mixture was stirred for 60 hours and then neutralised by addition of 2M hydrochloric acid. The mixture was loaded onto an Isolute® SCX cartridge, eluting with methanol, then 2M NH₃ in methanol. After evaporation of the basic fractions, the crude product was further purified by chromatography on silica gel (ethyl acetate/hexane). The product was dissolved in dichloromethane (3 mL) and then I MHCl in diethyl ether (1 mL) was added. After stirring for 1 hour, the solvents were evaporated to give the title compound. MS (ES+ve): [M+H]⁺ at m/z 377 (C₂₃H₂₈N₄O requires [M+H]⁺ at m/z 377).

### EXAMPLE 17

### Preparation of H+/K+ ATPase

The H+/K+ ATPase assay was based on Hongo et at (1990) Jpn J. Pharmacol. 52.295-305 “Purification and characterization of (H+,K+)-ATPase from hog gastric mucosa”

### Preparation of H+/K+ ATPase

Fresh porcine stomachs were obtained and washed with 0.9% NaCl. The surface mucus was removed by vigorously wiping; the fundic mucosa was then removed from the underlying muscular layer and suspended in a chilled 0.25M sucrose solution. Homogenization was carried out with polytron setting 5 for 3 minutes and the homogenate was centrifugated at 8,000 rpm for 15 minutes. The supernatants after filtration over stainless gauze were then centrifugated at 13,000 rpm for 15 minutes. The resulting supernatants were recentrifuged using rotor type 70 Ti at 31,000 rpm for 1 hour to obtain the crude microsomal sediment (F0). The crude microsomes were suspended in the 0.25M sucrose solution. The resuspended microsomes (4 mL, 11 mg/mL) were layered on a single step gradient made from 5 mL of 7% (w/v) Ficoll in the 0.25 sucrose solution and centrifugated using rotor type 41 Ti at 30,000 rpm for 40 minutes. The light membrane (FB) appeared at the interface of the 7% Ficoll, and the heavy membrane (FS) appeared in the form of a sediment. FB was collected and diluted to 10-fold with the 0.25M sucrose solution and then centrifugated using rotor type 41 Ti at 31,000 rpm for 1 hour.

The resulting sediments were resuspended in the 0.25M sucrose solution by 10 strokes of a loose-fitting motor-driven, Telfon pestle rotating at 1,000 rpm in a homogenizer and refrigerated overnight for the final purification.

The resuspended microsomes (8 mL/3.5 mg/mL) were furthermore layered on top of 5 mL of 7% (w/v) Ficoll in the 0.25M sucrose solution and centrifuged using rotor type 41 at 30,000 rpm for 40 minutes.

The heavy membrane (FBS), appearing in the form of a sediment, was then resuspended in the 0.25M sucrose solution and centrifugated using rotor type 41 Ti at 37,500 rpm for 2 hours.

The pellet was resuspended in 0.25M sucrose solution and stored at −80° C. until use.

Alternatively the protein can be prepared in the following procedure:
1. 4 pig stomachs obtained fresh from abattoir.
2. Stomachs excised and washed thoroughly with cold water, then shipped on ice saturated in phosphate buffered saline solution (PBS).
3. All subsequent operations are performed at 4° C.; the harvesting of material from the stomachs should be performed within a cold room (steps 2-6).
4. Fundic regions are removed and rinsed twice in ice-cold PBS.
5. The mucosa is peeled away from the stomach wall using a scalpel (it will tear off relatively easily and stay intact).
6. Liberally apply ice-cold saturated NaCl to the surface, leaving the stomachs upside-down in the NaCl solution. Leave to soak around 15 minutes.
7. After soaking vigorously wipe the mucosae with paper towels to remove all mucus and loose surface cells
8. Using a spoon, scrape the mucosa away from the underlying connective tissue from all 4 stomachs, and put it in 800 mL of 0.25M sucrose containing Sigma Cocktail protease inhibitors, 1 mM EDTA, 1 mM EGTA pH 7.4.
9. Using scissors, roughly homogenize the tissue as much as possible.
10. Dounce homogenize the suspension using a tight-fitting Teflon-glass homogenizer (e.g. 50 mL volume) with 6 strokes at maximum rpm.
11. Centrifuge the homogenate for 45 minutes at 20,000 g in a Beckman JA18 rotor at 4° C. Chill the tubes and rotor beforehand in the cold room (same for all subsequent centrifugation steps).
12. Decant off the supernatant from this spin and centrifuge further for 45 minutes at 100,000 g in a Beckman JA30.5 rotor.
13. Resuspend the pellets in a total of 171 mL of 0.25M sucrose containing Sigma Cocktail protease inhibitors: dounce homogenize as above. Keep on ice, and pool in one vessel.
14. Prepare discontinuous (step) gradients in tubes for a Beckman JS24.38 rotor (swing out type), consisting of 15 mL of 7% Ficoll (w/w) overlaid onto 5 mL of 34% (w/v) sucrose. Load carefully 16 mL of sample on the top.
15. Being careful not to disturb the tubes, centrifuge them for 2 h at 100,000 g in a JA24.38 rotor.
16. After centrifugation, There should be a clearly visible cloudy region at the Ficoll/0.25M sucrose interface. Collect this using a 1 mL Gilson pipette (try not to take sucrose—the material will be in the Ficoll and you should be able to harvest it all without disturbing the interface. Keep on ice
17. Pool all the samples together in one tube. Fill with 0.25M sucrose, and centrifuge for 45 minutes at 100,000 g in a Beckman JA30.5 rotor. Discard carefully the supernatant and resuspend the pellet in approximately 1 mL of 5 mM Pipes/Tris pH 7.4.
18. Transfer the material to a round bottom flask and shell-freeze (rotate flask at an angle in a bath of methanol and cardice, to form a thin “shell” of frozen material on inside of flask). Lyophilize immediately overnight.
19. Re-suspend the lyophilized material in a minimum volume of Pipes/Tris and freeze aliquots down at −80° C.

### EXAMPLE 18

### H+/K+ ATPase Assay

The H+/K+ ATPase activity was determined by spectrophotometric quantification of enzymatic inorganic phosphate release from ATP. Concentration response curve experiments were carried out from a starting concentration of test compounds of 100 μM with serial half log units dilution to 3 nM. One full curve contains 8 points in duplicate.

a) for determination of total ATPase activity 1 μL of the test compound was preincubated in 80 μL incubation assay buffer (37.5 mM Bis-Tris acetate, pH5.5, 4 mM MgCl₂, 10 mM KCl) and H+/K+ ATPase enzyme from example 17 (10 μL of 0.25 μg/mLmL) at 37° C. for 15 minutes.
b) for non-specific ATPase activity 1 μL of the test compound was preincubated in 80 μl control assay buffer (37.5 mM Bis-Tris acetate, pH5.5, 4 mM MgCl₂) an H+/K+ ATPase enzyme from example 17 (10 μL of 0.25 μg/mLmL) at 37° C. for 15 minutes. The reaction was initiated by adding 10 μL of 1 mM ATP to (a) and (b) and then incubating at 37° C. for 60 minutes.
Malachite green buffer was added 100 pt/well and absorbance was read at 630 nm. Specific H+/K+ ATPase activity is the total ATPase activity (in the presence of 10 mM KCl: reaction (a)) minus the basal, non-specific, ATPase activity (in the absence of KCl: reaction (b)).

Alternatively, the assay can be performed with the following slightly modified procedure:

Concentration response curve experiments were carried out from a starting concentration of test compounds of 100 μM with serial 4-fold dilutions. One full curve contains 11 points in duplicate.
a) for determination of total ATPase activity 0.14 of the test compound was preincubated in 10 μL incubation assay buffer (20 mM PIPES, pH6.0, 1 mM Mgcl₂, 10 mM KCl) and H+/K+ ATPase enzyme from example 17 (final assay concentration 0.25 μg/mL) at 37° C. for 15 minutes.
b) for non-specific ATPase activity 0.1 μl of the test compound was preincubated in 10 μl control assay buffer (20 mM PIPES, pH6.0, 1 mM MgCl₂) and H+/K+ ATPase enzyme from example 17 (final assay concentration 0.25 μg/mL) at 37° C. for 15 minutes.
The reaction was initiated by adding 10 μL of 0.2 mM ATP to (a) and (b) and then incubating at 37° C. for 60 minutes.
Malachite green buffer was added 30 μl/well and absorbance was read at 630 nm. Specific H+/K+ ATPase activity is the total ATPase activity (in the presence of 10 mM KCl: reaction (a)) minus the basal, non-specific, ATPase activity (in the absence of KCl: reaction (b)).
All compounds tested in one or other of the two methods described above had an IC₅₀ of <5 uM.

1-21. (canceled)

22. A compound of formula (I) 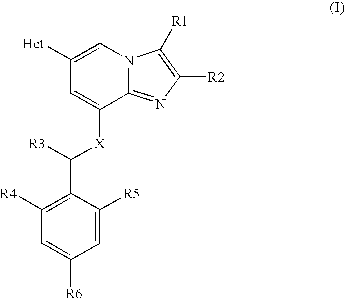
wherein
X is NH, NR7 or O;
R1 is H, C₁₋₄ alkyl, CH₂CN, CH₂NH₂, C₃₋₆cycloalkyl, C₃₋₆cycloalkylC₁₋₄alkyl, C₁₋₄alkoxy, C₂₋₆alkenyl, C₂₋₆alkenyloxyC₁₋₄alkyl, C₂₋₆alkynyl, hydroxyC₁₋₄alkyl, C₁₋₄alkoxyC₁₋₄alkyl, hydroxyC₁₋₄alkoxyC₁₋₄alkyl, fluoroC₁₋₄alkyl, C₂₋₆alkynyloxyC₁₋₄alkyl, C₁₋₄alkylsulphonylC₁₋₄alkyl, or NR8R9, where each of R8 and R9, which may be the same or different, are H or C₁₋₄alkyl or, together with the nitrogen to which they are attached, form a 5- or 6-membered heterocyclic group containing 0 to 3 further heteroatoms selected from the group consisting of N, O and S;
R2 is C₁₋₄alkyl, NH₂, C₃₋₆cycloalkyl, C₃₋₆cycloalkylC₁₋₄alkyl, C₁₋₄alkoxy, C₂₋₆alkenyl, hydroxyC₁₋₄alkyl, C₁₋₄alkoxyC₁₋₄alkyl, hydroxyC₁₋₄alkoxyC₁₋₄alkyl, cyanoC₁₋₄alkyl, haloC₁₋₄alkyl or aminocarboxyC₁₋₄alkyl;
R3 is H or C₁₋₄alkyl;
R4 and R5, which may be the same or different, are H, C₁₋₄alkyl, OH, halogen, C₁₋₄alkoxy; NR14R15 where each of R14 and R15, which may be the same or different, are H or C₁₋₄alkyl; NHCONR10R11, or OCONR10R11 where each of R10 and R11, which may be the same or different, are H or C₁₋₄alkyl or, together with the nitrogen to which they are attached, form a 5- or 6-membered heterocyclic group containing 0 to 3 further heteroatoms selected from the group consisting of N, O and S;
or R3 and R4 together with the interconnecting atoms form a 5- or 6-membered carbocyclic group or a heterocyclic group containing 1 heteroatom selected from the group consisting of N, O and S, which carbocyclic or heterocyclic group is optionally substituted with one group selected from C₁₋₄alkyl, OH, OC₁₋₄alkyl, halogen and NR16R17 where each of R16 and R17, which may be the same or different, are H or C₁₋₄alkyl;
R6 is H, C₁₋₄alkyl, halogen, OH, NHCO₂C₁₋₄alkyl, NR18R19 where each of R18 and R19, which may be the same or different, are H or C₁₋₄alkyl;
R7 is C₁₋₄alkyl;
or R4 and R7 together with the interconnecting atoms form a 5- to 7-membered heterocyclic group containing 0 or 1 further heteroatoms selected from N, O and S, which heterocyclic group is optionally substituted with one group selected from the group consisting of C₁₋₄alkyl, OH, OC₁₋₄alkyl, halogen and NR20R21 where each of R20 and R21, which may be the same or different, are H or C₁₋₄alkyl; and
Het is an optionally substituted 4 to 7-membered non-aromatic heterocyclyl group containing 1 to 3 heteroatoms selected from the group consisting of N, O and S;
or a pharmaceutically acceptable salt thereof.

23. A compound according to claim 22 wherein the Het group is attached to the imidazopyridine core through either a carbon atom or a heteroatom of the Het ring.

24. A compound according to claim 22 wherein the Het group is attached to the imidazopyridine core through the nitrogen atom of the Het ring.

25. A compound according to claim 22 wherein the Het group is selected from the group consisting of pyrrolidinyl, pyrrolidin-2-on-yl, dioxolanyl, imidazolinyl, imidazolidin-2-on-yl, oxazolidinyl, oxazolidin-2-on-yl, pyrazolidinyl, piperazinyl, ketopiperazinyl, diketopiperazinyl, piperidinyl, piperidin-2-on-yl, morpholinyl, thiomorpholinyl, morpholin-2-on-yl and isothiazolidinyl.

26. A compound according to claim 25 wherein Het is pyrrolidinon-yl, imidazolidinyl, piperidin-2-on-yl, imidazolidin-2-on-yl, morpholin-2-on-yl, or hydroxy-2-pyrrolidinon-yl.

27. A compound according to claim 26 wherein Het is piperidin-2-on-yl or imidazolidinonyl.

28. A compound according to claim 22 wherein the optional substituents on the Het group are (C₁₋₄)alkyl or hydroxy.

29. A compound according to claim 28 wherein (C₁₋₄)alkyl as optional substituents are methyl or ethyl.

30. A compound according to claim 22 wherein X is NH or O.

31. A compound according to claim 30 wherein X is NH.

32. A compound according to claim 30 wherein X is O.

33. A compound according to claim 22 wherein R1 and R2 are both methyl.

34. A compound according to claim 22 wherein R3 is H.

35. A compound according to claim 22 wherein R4 and R5 are both methyl, or R4 is methyl and R5 is ethyl.

36. A compound according to claim 22 wherein R6 is H.

37. A compound according to claim 22 wherein X is NH, R1 and R2 are both methyl, R3 is H, R4 and R5 are both methyl and R6 is H.

38. A compound according to claim 22 wherein X is O, R1 and R2 are both methyl, R3 is H, R4 and R5 are both methyl and R6 is H.

39. A compound according to claim 22 which is:
1-(8-{[(2,6-dimethylphenyl)methyl]amino}-2,3-dimethylimidazo[1,2-α]pyridin-6-yl)-2-pyrrolidinone hydrochloride;
1-(8-{[(2,6-dimethylphenyl)methyl]amino}-2,3-dimethylimidazo[1,2-α]pyridin-6-yl)-2-imidazolidinone hydrochloride;
N-[(2,6-dimethylphenyl)methyl]-2,3-dimethyl-6-(4-morpholinyl)imidazo[1,2-α]pyridin-8-amine hydrochloride;
N-[(2,6-dimethylphenyl)methyl]-2,3-dimethyl-6-(1-piperidinyl)imidazo[1,2-α]pyridin-8-amine hydrochloride;
N-[(2,6-dimethylphenyl)methyl]-2,3-dimethyl-6-(1-piperazinyl)imidazo[1,2-α]pyridin-8-amine hydrochloride;
1-(8-{[(2,6-dimethylphenyl)methyl]amino}-2,3-dimethylimidazo[1,2-α]pyridin-6-yl)-2-piperidinone hydrochloride;
1-(8-{[(2,6-dimethylphenyl)methyl]amino}-2,3-dimethylimidazo[1,2-α]pyridin-6-yl)-3-methyl-2-imidazolidinone hydrochloride;
1-(8-{[(2,6-dimethylphenyl)methyl]oxy}-2,3-dimethylimidazo[1,2-α]pyridin-6-yl)-2-piperidinone;
N-[(2,6-dimethylphenyl)methyl]-6-[cis-3,5-dimethyl-1-piperazinyl]-2,3-dimethylimidazo[1,2-α]pyridin-8-amine hydrochloride;
N-[(2,6-dimethylphenyl)methyl]-6-(1,1-dioxido-2-isothiazolidinyl)-2,3-dimethylimidazo[1,2-α]pyridin-8-amine hydrochloride;
4-(8-{[(2,6-dimethylphenyl)methyl]amino}-2,3-dimethylimidazo[1,2-α]pyridin-6-yl)-3-morpholinone hydrochloride;
(4S)-1-(8-{[(2,6-dimethylphenyl)methyl]amino}-2,3-dimethylimidazo[1,2-α]pyridin-6-yl)-4-hydroxy-2-pyrrolidinone hydrochloride;
(4R)-1-(8-{[(2,6-dimethylphenyl)methyl]amino}-2,3-dimethylimidazo[1,2-α]pyridin-6-yl)-4-hydroxy-2-pyrrolidinone hydrochloride;
(4S)-1-(8-{[(2,6-dimethylphenyl)methyl]oxy}-2,3-dimethylimidazo[1,2-α]pyridin-6-yl)-4-hydroxy-2-pyrrolidinone hydrochloride;
4-(8-{[(2,6-dimethylphenyl)methyl]oxy}-2,3-dimethylimidazo[1,2-α]pyridin-6-yl)-3-morpholinone hydrochloride;
1-(8-{[(2-ethyl-6-methylphenyl)methyl]amino}-2,3-dimethylimidazo[1,2-α]pyridin-6-yl)-2-pyrrolidinone hydrochloride;
or other salts thereof.

40. A method of treating or preventing duodenal ulcer, gastritis, gastro-oesophageal reflux disease (GERD), and Zoller-Ellison Syndrome which method comprises administering an effective amount of a compound of formula 1 according to claim 22, either alone or combined with a pharmaceutically acceptable carrier.

41. A pharmaceutical composition comprising a compound of formula (I) as defined in claim 22 or a pharmaceutically acceptable salt thereof, and a pharmaceutically acceptable carrier.
